(19) United States
(12) Patent Application Publication
(10) Pub. No.: US 2017/0305849 A1
(43) Pub. Date: Oct. 26, 2017

(54) METHOD FOR PRODUCING ASTAXANTHIN ESTERS

(71) Applicant: BASF SE, Ludwigshafen (DE)

(72) Inventors: Bernd SCHÄFER, Dierbach (DE); Stefan BENSON, Bensheim (DE); Wolfgang SIEGEL, Limburgerhof (DE)

(21) Appl. No.: 15/509,905

(22) PCT Filed: Aug. 11, 2015
(86) PCT No.: PCT/EP2015/068445

§ 371 Date: Mar. 9, 2017

(30) Foreign Application Priority Data
Sep. 11, 2014 (EP) 14184483.7

(51) Int. Cl. C07C 403/24; C07C 2601/16

(52) U.S. Cl. CPC C07C 403/24; C07C 2601/16

(57) ABSTRACT

The invention describes an environmentally friendly, sustainable and cost-effective method for preparing astaxanthin diesters of the formula 1, in which astaxanthin of the formula 2 is doubly esterified with fatty acid chlorides of the general formula 3. For this purpose, compound 2 and 3 are reacted in an organic solvent in the presence of a nitrogen-containing base of the general formula 4. The invention further relates to the non-therapeutic use of the diester 1, in which R is a residue selected from the group consisting of C13-C19-alkyl, C13-C19-alkenyl, C13-C19-alkdienyl and C13-C19-alktrienyl, in human or animal nutrition and also the therapeutic use of the diester 1 prepared according to the method as a medicament and also as an ingredient in a medicinal preparation.

The present invention relates to a method for preparing an astaxanthin diester and the use thereof.

Industrial syntheses of astaxanthin have been described in detail both in the relevant literature, e.g. G. Britton, S. Liaanen-Jensen, H. Pfander, Carotenoids, Vol. 2, Birkhäuser Verlag, Basle, 1996, 283 ff., and in various textbooks, e.g. B. Schäfer, Naturstoffe der chemischen Industrie (Natural Substances of the Chemical Industry), Akademischer Verlag, Heidelberg, 2007, 427 ff., in scientific journals, e.g. K. Meyer, Chemie in unserer Zeit (Chemistry in Our Time) 36 (2002) 178 and also in the patent literature, e.g. DE 10049271 (2000) or EP 1285912 (2003).

Numerous astaxanthin diesters have also already been described to date. They generally take the form of diesters bearing often further O-, S- and N-containing functional groups in the acid residue. Examples include astaxanthin diethylsuccinate, astaxanthin di(3-methylthiopropionate) and astaxanthin dinicotinate (WO 2003/066 583 A1, WO 2011/095 571). According to the teaching of these documents, astaxanthin is reacted with acids, acid chlorides or acid anhydrides in the presence of coupling reagents such as ethyl chloroformate or N,N-dicyclohexylcarbodiimide, or bases such as triethylamine or pyridine, and catalysts such as DMAP.

Interestingly, in the case of fatty acid esters of astaxanthin (which are understood to mean, in the broadest sense, carboxylic acid residues without further O-, S- and N-containing functional groups), only enzymatic esterifications using lipases are currently known, particularly with mid-range fatty acids (comprising 8 to 12 C atoms, (M. Nakao, M. Sumida, K. Katano, H. Fukami, J. Oleo Sci. 57 (2008) 371).

An exception is a fatty acid ester of astaxanthin, which is obtained, according to the teaching of the Spanish patent ES 2223270, by esterifying zeaxanthin and then oxidizing this ester with pyridinium chlorochromate. Specifically, the dipalmitate is prepared, starting from zeaxanthin, and the corresponding astaxanthin dipalmitate is obtained therefrom by oxidation.

Although it would mean one fewer method step and therefore it would be quicker and consider-ably more cost-effective, the person skilled in the art in ES 2 223 270 does not proceed directly from astaxanthin as starting material but from zeaxanthin in order to prepare astaxanthin dipalmitate. Accordingly, it was not obvious to a person skilled in the art even in 2003 to prepare, for example, astaxanthin dipalmitate directly from astaxanthin and, in particular, to prepare astaxanthin dipalmitate directly from astaxanthin without costly oxidizing agents and/or coupling reagents.

The majority of the result of the work of the applicant tend in the same direction, as further shown in the comparative examples below, where many experiments to prepare long-chain fatty acid diesters of astaxanthin directly from astaxanthin afforded only very low, if any, yields.

Moreover, it was found in the low yields recorded that in the majority of cases they were obtained only after very long, and therefore uneconomic, reaction times.

The following also refers to the fact that the corresponding astaxanthin diesters cannot readily be prepared from long-chain fatty acid units and astaxanthin in a cost-effective and time-saving manner. It has been known since 1982 that astacin, with the formula A below,

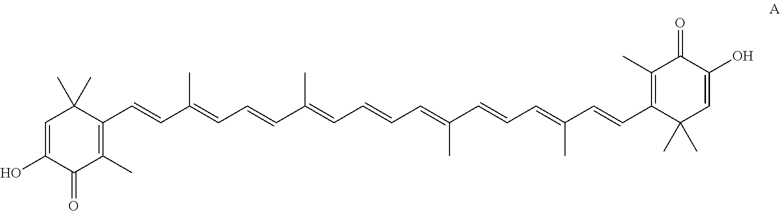 [SMILES: CC1=C(/C=C/C(C)=C/C=C/C(C)=C/C=C/C=C(C)/C=C/C=C(C)/C=C/C2=C(C)C(=O)C(O)=CC2(C)C)C(C)(C)C=C(O)C1=O]

can be converted into the corresponding diester using a fatty acid chloride. It is stated in the article of Widmer at al. in Helv. Chim. Acta. 65(3) 1982 671 on p. 683 In example 8: “Preparation of astacin dipalmitate (29). By reaction of 3.3 g of astacin 1 (5.6 mmol) with 3.4 g of palmitoyl chloride (12.2 mmol) in 50 ml of pyridine (45″; 4 h) and work-up with 700 ml of 1.7 N H₂SO₄, 400 ml of CH₂Cl₂ and 100 ml of sat. aqueous NaHCO₃ solution, a crude product was obtained, . . . : 5.0 g (83.5%) of 29 as red-violet, somewhat sticky crystals;”

Astacin of the formula A differs structurally from astaxanthin of the formula 2 below

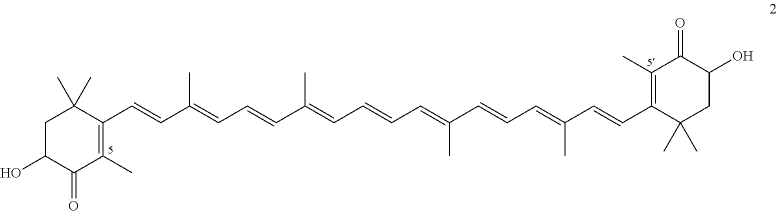 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

only in that the latter compound comprises only one cyclic double bond, while astacin of the formula A has two double bonds per cycle. Accordingly, from this starting point, it would be simple for a person skilled in the art to use the teaching for the preparation of astacin esters from astacin also to form corresponding astaxanthin esters from astaxanthin.

The applicant, however, could not find information of this kind in the prior art. Instead, procedures have been selected from the Spanish document already mentioned above in order to obtain a fatty acid diester of astaxanthin.

A technical object of the invention to be achieved arising therefrom is to overcome the disadvantages of the prior art and to find a generally valid, simple method for esterifying astaxanthin using moderate and long-chain fatty acids (from C9 to C20). Said method shall also be applicable to large amounts of reactant, but nevertheless be energy efficient. Moreover, it should be cost-effective, i.e. it does not require expensive coupling reagents, and should afford high yields of diester. It should, moreover, rapidly produce the desired diester, i.e. it should reduce and, as far as possible, avoid excess reaction or method steps and be characterized by high reaction rates. In addition, by-products should as far as possible hardly occur, if at all, and, if unavoidable, be readily removable. Solvents used should be removable from the reaction mixture with minimum effort and be re-usable. In addition, the proportion of water-polluting substances, which are readily miscible with water and therefore generally difficult to remove, should be reduced. Furthermore, the aim is to obtain the diester of astaxanthin in high yield as far as possible as a solid or crystalline solid using moderate and long-chain fatty acids (from C9 to C20).

Main features of the invention are the subject matter of claims 1, 16 and 17. Further configurations arise from claims 2 to 15.

Thus, an astaxanthin diester of the general formula 1

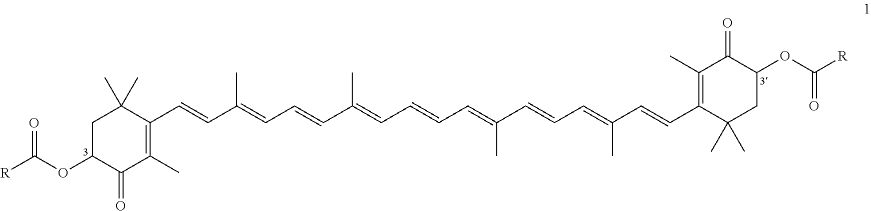

in which the asymmetric center in position 3 and 3′ is racemic, or each has (S) or (R) configuration and R is a residue selected from the group consisting of C9-C19-alkyl, C9-C19-alkenyl, C9-C19-alkdienyl and C9-C19-alktrienyl, is obtained by a preparation method according to the invention, in which astaxanthin of the formula 2

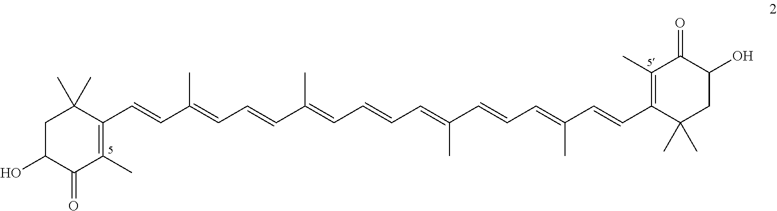 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

in an organic solvent is reacted with an acid chloride of the general formula 3

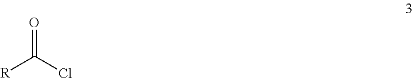

in which R is as defined in formula 1, in the presence of at least one nitrogen-containing base of the general formula 4

NR¹R²R³4

in which R¹, R² and R³ are each independently selected from the group consisting of a saturated C1-C6 chain, an unsaturated C1-C6 chain, an aromatic C6 ring, a C1-C6 chain formed from two of the three residues R¹, R² and R³, wherein said two residues are linked to each other and, together with the nitrogen atom of the base 4, form an alkylated or non-alkylated heterocycle or an alkylated or non-alkylated heteroaromatic cycle, or a C1-C6 chain formed from two of the three residues R¹, R² and R³, wherein said two residues are linked to each other via a further nitrogen atom and, together with the nitrogen atom of the base 4, form an alkylated or non-alkylated heterocycle or an alkylated or non-alkylated heteroaromatic cycle.

This result was not readily predictable. Firstly, the prior art provides no references to this, as already stated above.

Secondly, astaxanthin of the formula 2 and astacin of the formula A are completely different in terms of their reactivity. Therefore, the esterification of astaxanthin of the formula 2 and of astacin of the formula A presents two basically different aspects which, to a person skilled in the art, are to be found essentially in the steric environment of the six-membered ring system.

Whereas in astaxanthin of the formula 2 only 3 C atoms are sp² hybridized, namely those in positions 4, 5 and 6, in astacin of the formula A no fewer than 5 C atoms are sp² hybridized, namely those in positions 2, 3, 4, 5 and 6. The distorted chair conformation of astaxanthin of the formula 2 is thereby substantially flattened and in astacin of the formula A is more equal to that of benzene (which has 6 sp² hybridized C atoms). In the case of astaxanthin of the formula 2, a person skilled in the art expects a distinct steric effect by the two methyl groups in position 1 on the reactivity of the hydroxyl group, due to a 1,3-transannular interaction, which is included in the standard repertoire of every textbook of organic chemistry, especially in regard to six-membered ring systems. Due to the flattening of the six-membered ring in the case of astacin of the formula A, this esterification-disrupting interaction is negated such that the esterificatons are more readily possible and a formal comparison of the two molecules, astaxanthin of the formula 2 and astacin of the formula A, in terms of the objective according to the invention, is not valid.

A person skilled in the art would have expected, according to that stated above, that a reaction of astaxanthin with the claimed acid chlorides in the presence of various bases to give the corresponding diester is impossible or barely possible. Not just this is strikingly confirmed as further illustrated below. In fact, even non-chloride-activated fatty acids having 9 to 19 C atoms show little or no tendency to form a corresponding diester with astaxanthin of the formula 2. For example, if vinyl palmitate is added to astaxanthin in the presence of Novozyme 435 (CAS number 9001-62-1), no reaction is observed at all, as is likewise further illustrated below in the relevant comparative example. If in the comparative examples any reaction could be recorded, then it is generally incomplete and after a very long reaction time.

Moreover, example 8 of the Widmer article is conducted in pyridine. This compound is thus concentrated, i.e. used simultaneously as solvent and nitrogen-containing base. In view of the poor comparability of astacin and astaxanthin described above, a person skilled in the art would have just exchanged astacin for astaxanthin, in analogy to Widmer, but would otherwise have chosen exactly the same reaction conditions in the hope of achieving any conversion to the corresponding diester. Therefore, said person skilled in the art would have worked in concentrated pyridine, knowing the poor reactivity of astaxanthin, in order to achieve in the best case a roughly acceptable esterification of this molecule in analogy to Widmer.

It is therefore all the more surprising that, in accordance with the invention, good results are achieved in an organic solvent where this solvent does not comprise any nitrogen-containing base, as further illustrated below. The latter is only added in molar amounts which vary in the range of the corresponding molar amounts of the acid chloride used and at most account for a 3-fold molar excess with respect to the acid chloride.

Accordingly, the method according to the invention differs from Widmer in two essential features: 1. In place of astacin of the formula A, astaxanthin of the formula 2 is used for the conversion to a corresponding diester. 2. The solvent used is an organic solvent instead of pyridine.

The fact that, despite the discouraging results in the comparative experiments, astaxanthin can be reacted with an acid chloride to give the corresponding diester in good yields and after short reaction times and this is possible even in an organic solvent and not exclusively in pure pyridine, is astonishing and this was astounding to the applicant.

Since acid chlorides of the general formula 3 and nitrogen-containing bases of the general formula 4 are much less expensive to acquire than coupling reagents with which the corresponding free acids of the acid chlorides of the general formula 3 have to be activated before reaction with astaxanthin of the formula 2, the method according to the invention is also advantageous from an economic point of view and applicable on an industrial scale.

Moreover, the pyridine used as solvent by Widmer readily dissolves in water and therefore ends up in the aqueous phase on work-up and has to be removed therefrom as water-polluting material. If pyridine is no longer to be used as solvent, its removal is in large parts or even completely avoided, whereby the method according to the invention is more economical and environmentally friendly.

The term “racemic”, as used in claim 1, signifies that the stereochemistry at position 3 and 3′ is arbitrary. The term “(S)-configuration” is understood to mean that an arrangement of the individual substituents at position 3 and 3′ is such that the numbering, going from the heaviest substituent around to the lightest substituent, is counterclockwise, i.e. to the left, whereas in the term “(R)-configuration” it is clockwise, i.e. to the right. The numbering in both cases is based on the lightest substituent facing away from the viewer while counting.

R comprises the residues C9-C19-alkyl, C9-C19-alkenyl, C9-C19-alkdienyl, C9-C19-alktrienyl. C9-C19-alkyl is understood to mean all those residues comprising at least 9 and at most 19 saturated carbon atoms. C9-C19-alkyl is preferably understood to mean all those residues comprising at least 9 and at most 19 saturated carbon atoms linked to one another in linear fashion. C9-C19-alkyl is accordingly selected from the group consisting of n-nonyl or n-pelargonyl, n-decyl or n-capryl, n-undecyl, dodecyl or n-lauryl, n-tridecyl, n-tetradecyl or n-myristyl, n-pentadecyl, n-hexadecyl or n-palmityl, n-heptadecyl, n-octadecyl or n-stearyl and n-nonadecyl.

C9-C19-alkenyl is understood to mean all those residues comprising at least 9 and at most 19 carbon atoms, in which two of them are linked to each other via a double bond with E or Z configuration. C9-C19-alkenyl is preferably understood to mean all those residues comprising at least 9 and at most 19 carbon atoms linked to one another in linear fashion, in which two of them are linked to each other via a double bond with E or Z configuration. C9-C19-alkenyl is accordingly selected from the group consisting of n-nonenyl, n-decenyl, n-undecenyl, n-dodecenyl, n-tridecenyl, n-tetradecenyl, n-pentadecenyl, n-hexadecenyl, for example (9Z)-n-hexadec-9-enyl or palmitoleyl, n-heptadecenyl, n-octadecenyl, for example (9Z)-n-octadec-9-enyl or oleyl, (9E)-n-octadec-9-enyl or elaidinyl and n-nonadecenyl.

C9-C19-alkdienyl is understood to mean all those residues comprising at least 9 and at most 19 carbon atoms, in which said residues have two double bonds with E and/or Z configuration. C9-C19-alkdienyl is preferably understood to mean all those residues comprising at least 9 and at most 19 carbon atoms linked with one another in linear fashion, in which said residues have two double bonds with E and/or Z configuration. C9-C19-alkdienyl is accordingly selected from the group consisting of n-nonadienyl, n-decadienyl, n-undecadienyl, n-dodecadienyl, n-tridecadienyl, n-tetradecadienyl, n-pentadecadienyl, n-hexadecadienyl, n-heptadecadienyl, n-octadecadienyl, for example [(9Z,12Z)-octadeca-9,12-dienyl or linoleyl and n-nonadecadienyl.

C9-C19-alktrienyl is understood to mean all those residues comprising at least 9 and at most 19 carbon atoms, in which said residues have three double bonds with E and/or Z configuration. C9-C19-alktrienyl is preferably understood to mean all those residues comprising at least 9 and at most 19 carbon atoms linked with one another in linear fashion, in which said residues have three double bonds with E and/or Z configuration. C9-C19-alktrienyl is accordingly selected from the group consisting of n-nonatrienyl, n-decatrienyl, n-undecatrienyl, n-dodecatrienyl, n-tridecatrienyl, n-tetradecatrienyl, n-pentadecatrienyl, n-hexadecatrienyl, n-heptadecatrienyl, n-octadecatrienyl, for example (9Z,12Z,15Z)-octadeca-9,12,15-trienyl or linolenyl, (6Z,9Z,12Z)-octadeca-6,9,12-trienyl or gamma linolenyl, (9Z,11E,13E)-octadeca-9,11,13-trienyl or elaeostearyl, (5Z,9Z,12Z)-octadeca-5,9,12-trienyl or pinolenyl, (5E,9Z,12Z)-octadeca-5,9,12-trienyl or columbinyl, n-nonadecatrienyl, (8Z,11Z,14Z)-eicosa-8,11,14-trienyl or dihomo-gamma-linolenyl.

C9-C19-alktrienyl further comprises the alkyl residue of arachidonic acid, i.e. a residue comprising 19 C atoms and four double bonds (formally a C19-alktetraenyl residue but which has also been included under the term “C9-C19-alktrienyrl” for the sake of easier readability).

Suitable solvents for the method according to the invention are all organic solvents in which astaxanthin and the relevant reaction partners are sufficiently readily soluble. The organic solvent therefore comprises at least one compound selected from the group consisting of dichloromethane, ethylene glycol dimethyl ether, diethylene glycol dimethyl ether, tetrahydrofuran, ethylene carbonate, propylene carbonate, dimethylformamide, dimethyl sulfoxide, ethyl acetate, n-propyl acetate, toluene, xylene, heptane, hexane, pentane, N-methyl-2-pyrrolidone, dioxane, 2-methyltetrahydrofuran, methyl tert-butyl ether, diisopropyl ether, diethyl ether, di-n-butyl ether, acetonitrile, trichloromethane, chlorobenzene and preferably from the group consisting of dichloromethane, trichloromethane, ethylene glycol dimethyl ether, diethylene glycol dimethyl ether, tetrahydrofuran, chlorobenzene, ethylene carbonate, propylene carbonate, ethyl acetate and methyl tert-butyl ether. In the context of this disclosure, nitrogen-containing bases, in particular pyridine, are explicitly not included in the organic solvents according to the invention.

Acid chlorides according to the invention are all those compounds R—C(═O)Cl of the formula 3, in which R is a residue selected from the group of C9-C19-alkyl, C9-C19-alkenyl, C9-C19-alkdienyl and C9-C19-alktrienyl, as defined above.

“Nitrogen-containing base of the general formula 4” is understood to mean all bases comprising at least one nitrogen atom, and also that the residues R¹, R², R³ form a hydrochloride with hydrogen chloride (HCl). Amides are not included under the term “nitrogen-containing base”.

In accordance with the invention, a “saturated C1-C6 chain” is selected from the group consisting of methyl, ethyl, n-propyl, isopropyl, n-butyl, sec-butyl, isobutyl, tert-butyl, n-pentyl, n-hexyl, cyclopentyl and cyclohexyl.

In accordance with the invention, an “unsaturated C1-C6 chain” is selected from the group consisting of vinyl, allyl, prenyl, isoprenyl, homoallyl, cyclopentadienyl and cyclohexenyl.

In accordance with the invention, an “aromatic C6 ring” is phenyl.

A continuation of the method according to the invention provides that the astaxanthin of the formula 2 in the organic solvent is reacted with a greater than two-fod molar excess, based on astaxanthin 2, of the acid chloride of the general formula 3 in the presence of at least one nitrogen-containing base of the general formula 4. It is generally sufficient to use double the amount of acid chloride of the general formula 3 per mole of astaxanthin of the formula 2, as there are no further reactive groups accessible to the acid chloride 3 besides the two OH groups of the astaxanthin 2. A person skilled in the art would not in any case use larger amounts for reasons of cost. It has been found, however, based on experiments in the context of this invention, that technical grade acid chloride is never completely free of the corresponding free carboxylic acids, particularly when operating with larger batches or In continuous operation. Such traces of free carboxylic acid have the effect however that a certain portion of the acid chloride of the general formula 3 forms the corresponding anhydride with the free carboxylic acid. The latter accumulates in the reaction mixture but no longer reacts with astaxanthin of the formula 2. In order nevertheless to achieve the best possible conversion of astaxanthin of the formula 2 with the corresponding acid chloride of the general formula 3, this continuation of the method according to the invention is therefore of particular significance.

A further refined configuration of the method according to the invention provides that the astaxanthin of the formula 2 in the organic solvent is reacted with a 2.1-fold to 9-fold molar excess, based on astaxanthin, preferably with a 2.3-fold to 7-fold molar excess, more preferably with a 2.5-fold to 5-fold molar excess and most preferably with a 2.7-fold to 3-fold molar excess, of the acid chloride of the general formula 3 in the presence of at least one nitrogen-containing base of the general formula 4. The amount of acid chloride of the general formula 3 used, according to the embodiments stated above, should be sufficiently large that losses caused by hydrolysis and by anhydride formation are compensated for and at least 2 moles of reactive acid chloride of the general formula 3 are available per mole of astaxanthin of the formula 2. On the other hand, use of too large amounts of acid chloride of the formula 3 not only drives up the costs of the method according to the invention, but also a larger amount of undesired anhydride of the acid chloride of the formula 3 is inevitably formed. High conversion with simultaneous minimal anhydride formation could be achieved with the concentrations of acid chloride of the general formula 3 mentioned above and, for this reason, this further refined configuration of the method according to the invention is also of significance.

A further aspect of the invention provides that astaxanthin of the formula 2 in a chlorine-containing organic solvent is reacted with the acid chloride of the general formula 3 in the presence of at least one nitrogen-containing base of the general formula 4, preferably in a chlorine-containing organic solvent selected from the group consisting of dichloromethane, trichloromethane, tetrachloromethane, 1,1-dichloroethane, 1,2-dichloroethane, trichloroethylene, tetrachloroethylene, perchloroethylene, chlorobenzene or a mixture of at least two of these solvents.

Preference is given to using chlorine-containing solvents such as dichloromethane, trichloromethane or chlorobenzene or a mixture of these solvents. Xantophylls and also β-carotene itself are typically only moderately soluble or insoluble in solvents. This is also confirmed by Widmer on p. 678 in the last paragraph of the publication Helv. Chim. Acta. 65(3) 1982 671, in which he writes: “It was thus once more demonstrated that chemical reactions on carotenoids already built up to the C₄₀ stage may often be linked to major problems, especially as the purification of the resulting mixtures is also difficult”. Low solubility is generally detrimental, however, for a reaction in a liquid medium or in solution. In the abovementioned solvents, despite the generally poor solubility of astaxanthin of the formula 2, good conversions and yields were achieved. Moreover, the non-aromatic solvents mentioned are characterized in that they can be removed at a low temperature and standard pressure due to their low boiling point. Chlorobenzene can also be readily removed under reduced pressure or by extraction from the other components of the reaction mixture due to its high hydrophobicity. Finally, all solvents mentioned in this and in the previous paragraph are immiscible with water, and to this extent a costly water treatment is avoided. This aspect of the method is therefore also of significance in terms of the invention. The method according to the invention should be, inter alia, energy efficient and cost-effective in comparison with the prior art. This aim is achieved if the astaxanthin of the formula 2 in the organic solvent is reacted with the acid chloride of the general formula 3 in the presence of at least one nitrogen-containing base of the general formula 4 in a temperature range of −20 to +100° C., particularly in a temperature range of 0° C. to 60° C. This means that the reaction according to the invention is carried out in a temperature range of −20 to +100° C., particularly in a temperature range of 0° C. to 60° C.

If the examples and comparative examples given below are considered in summary, it is evident that a complete conversion of astaxanthin of the formula 2 to the diester of the formula 1 is possible in the presence of cyclic nitrogen-containing bases. Therefore, a continuation of the invention specifies that astaxanthin of the formula 2 in the organic solvent is to be reacted with the acid chloride of the general formula 3 in the presence of at least one nitrogen-containing base of the general formula 4, in which the base 4 is selected from the group consisting of monocyclic nitrogen-containing bases, preferably pyridines or imidazoles and bicyclic nitrogen-containing bases such as DBU.

The bases used are preferably monocyclic nitrogen-containing bases such as pyridines, particularly pyridine, 4-dimethylaminopyridine, 3-methylpyridine and 5-ethyl-2-methylpyridine or imidazoles such as N-methylimidazole or bicyclic nitrogen-containing bases such as DBU.

Monocyclic nitrogen-containing bases are selected from the group comprising aziridines, azetidines, pyrroles, pyrrolidines, pyrrazoles, imidazoles, triazoles, tetrazoles, pyridines, pyridazines, pyrimidines, pyrazines, triazines and tetrazines.

Bicylic nitrogen-containing bases are selected from the groups comprising indoles, quinolines, isoquinolines, purines, 1,8-diazabicyclo[5.4.0]undec-7-ene (DBU), 1,5-diazabicyclo[4.3.0]non-5-ene, 1,4-diazabicyclo[2.2.2]octane and 4-(N-pyrrolidinyl)pyridine.

The nitrogen-containing base of the general formula 4 is particularly preferably selected from the group consisting of N-methylimidazole, 2-methylimidazole, 4-methylimidazole, pyridine, 3-methylpyridine, 2-methylpyridine, 4-methylpyridine, 4-dimethylaminopyridine, 5-ethyl-2-methylpyridine and nicotine, since complete reaction of the acid chloride of the general formula 3 with astaxanthin of the formula 2 to give the corresponding astaxanthin diester of the general formula 1 is possible with these nitrogen-containing bases.

Therefore, a significant embodiment of the method according to the invention provides that astaxanthin of the formula 2 in the organic solvent is reacted with the acid chloride of the general formula 3 in the presence of at least one nitrogen-containing base of the general formula 4, in which the base 4 is selected from the group consisting of N-methylimidazole, 2-methylimidazole, 4-methylimidazole, pyridine, 3-methylpyridine, 2-methylpyridine, 4-methylpyridine, 4-dimethylaminopyridine, 4-(N-pyrrolidinyl)pyridine, 5-ethyl-2-methylpyridine and nicotine.

Not only a complete, but also a quite prompt conversion to the diester 1 is achieved if the astaxanthin of the formula 2 in the organic solvent is reacted with the acid chloride of the general formula 3 in the presence of at least one nitrogen-containing base of the general formula 4, in which the base 4 is selected from the group consisting of N-methylimidazole, pyridine, 3-methylpyridine, 4-dimethylaminopyridine and 5-ethyl-2-methylpyridine.

The compound 1,1′-carbonyldiimidazole (CDI) is not, however, to be included in the cyclic nitrogen-containing bases since it is an activating reagent for a carboxylic acid (see comparative examples below).

The nitrogen-containing bases of the general formula 3 are generally water-soluble, but also dissolve partially in the organic solvent or precipitate as hydrochloride. Therefore, complete removal from the reaction mixture is then particularly difficult if said bases are used in amounts which far exceed that required for the reaction procedure. To avoid this, a further aspect of the invention provides that the astaxanthin of the formula 2 in the organic solvent is reacted with the acid chloride of the general formula 3 in the presence of at least one nitrogen-containing base of the general formula 4, in which the base is used in a 1 to 3-fold molar ratio, preferably in a 1.1 to 2-fold molar ratio and most preferably in a 1.1 to 1.5-fold molar ratio, based on the acid chloride of the general formula 3. With these amounts it is ensured that, firstly, the hydroxyl groups of astaxanthin of the formula 2 are catalytically deprotonated, forming HCl which is bound as the hydrochloride and, secondly, not so much base is present in the reaction mixture such that it can only be removed with difficulty. As such, a considerable improvement compared to example 8 from Helv. Chim. Acta. 65(3) 1982 671 is achieved, which allows for reaction of astacin A and not astaxanthin 2 in pure pyridine as solvent.

As already implied above, an operation without traces of free carboxylic acid, which is desirable for esterifications with an acid chloride, cannot be ensured in long term or continuous operation, particularly with large amounts of starting compound astaxanthin of the formula 2. Traces of said free carboxylic acid, however, on reaction with further acid chloride of the general formula 3, lead to the formation of the corresponding anhydrides, which no longer react with astaxanthin of the formula 2 and remain in the reaction mixture. These can only be removed therefrom with difficulty. They are also still present in traces in the diester 1 according to the invention, which is why these can be obtained after purification only as oils and not as solids.

An essential further elaborated variant of the method according to the invention therefore aims to resolve this deficiency. This specifies that astaxanthin of the formula 2 in the organic solvent is reacted with the acid chloride of the general formula 3 in the presence of at least one nitrogen-containing base of the general formula 4; and that the resulting reaction mixture is treated with at least one compound selected from the group consisting of alcohols of the general formula 5: R⁴OH where R⁴ is equal to C1-C6-alkyl and amines of the general formula 6: R⁵R⁶NH where R⁵ and R⁶ are each independently equal to H or C1-C6-alkyl, in which R⁵ and R⁶ either each form an independent group or are linked to each other.

In other words, it can also be said that the addition in the course of the work-up of alcohols of the general formula 5 R⁴OH, where R⁴ is equal to C1-C6-alkyl, is advantageous, since potential by-products can be more easily removed. Methanol, ethanol and n-propanol have proven to be particularly advantageous. It is likewise advantageous during the course of the work-up to use amines of the general formula 6 R⁵R⁶NH, where R⁵ and R⁶ are each independently equal to H or C1-C6-alkyl, which also includes R⁵ and R⁶ linked to each other.

The residues R⁵ and R⁶ are selected from the group consisting of H and C1-C6-alkyl. The residue R⁴ includes all those moieties which can be incorporated under the term C1-C6-alkyl. The term C1-C6-alkyl includes all those moieties selected from the group consisting of methyl, ethyl, n-propyl, isopropyl, n-butyl, sec-butyl, isobutyl, tert-butyl, pentyl, n-hexyl, cyclopentyl and cyclohexyl.

If the resulting reaction mixture, i.e. the reaction mixture after completion of the esterification reaction, is treated with at least one compound selected from alcohols of the general formula 5 and amines of the general formula 6, the corresponding ester and/or corresponding amide is formed from excess acid chloride of the general formula 3 as well as from the anhydrides formed. Both amides and esters of the acid chloride of the general formula 3 can be more easily removed from the reaction mixture in contrast to the anhydride mentioned above. It is possible by this measure to isolate diester of the formula 1 in a simple manner, even as a solid.

A particularly preferred variant of the method according to the invention relates therefore to re-acting the astaxanthin of the formula 2 in dichloromethane, trichloromethane, chlorobenzene or a mixture of at least two of these organic solvents, with the acid chloride of the general formula 3 in the presence of at least one nitrogen-containing base selected from the group consisting of N-methylimidazole, pyridine, 3-methylpyridine, 4-dimethylaminopyridine and 5-ethyl-2-methylpyridine; and to treating the resulting reaction mixture with at least one compound selected from the group consisting of alcohols of the general formula 5: R⁴OH where R⁴ is equal to C1-C6-alkyl and amines of the general formula 6: R⁵R⁶NH where R⁵ and R⁶ are each independently equal to H or C1-C6-alkyl, in which R⁵ and R⁶ either each form an independent group or are linked to each other.

If, on completion of the esterification according to the invention, amines of the general formula 6 or alcohols of the general formula 5 are added in excess salts may be formed. These salts must be removed from the reaction product. Moreover, certain alcohols, such as, inter alia, methanol, tend to partition in a biphasic mixture both into the polar phase and into the hydrophobic or organic phase. Compounds, which are readily soluble in methanol for example, are then likewise distributed in both phases and this results in an incomplete, therefore undesired, separation of these compounds into one phase.

These disadvantages may be countered with the following extensions of the method according to the invention. This comprises astaxanthin of the formula 2 in the organic solvent being reacted with the acid chloride of the general formula 3 in the presence of at least one nitrogen-containing base of the general formula 4; and the resulting reaction mixture being treated with a molar deficiency, based on the amount of acid chloride 3, of at least one compound selected from the group consisting of alcohols of the general formula 5 and amines of the general formula 6.

If the acid chloride 3, with respect to the amount, is used with a molar deficiency of at least one compound selected from the group consisting of alcohols of the general formula 5 and amines of the general formula 6, this compound initially reacts with excess acid chloride of the formula 3 and with partially formed anydrides thereof to give the corresponding esters or amides. Therefore, the compound of the formula 5 and/or 6 is, to a large extent, or even completely, consumed and can no longer lead to mixture phenomena described above.

As is evident from the examples below, a method procedure has proven to be particularly practicable in which astaxanthin of the formula 2 in the organic solvent is reacted with the acid chloride of the general formula 3 in the presence of at least one nitrogen-containing base of the general formula 4; and the resulting reaction mixture is treated with a 0.1 to 0.9-fold molar 20 amount, based on the amount of acid chloride 3, preferably with a 0.2 to 0.7-fold molar amount, more preferably with a 0.3 to 0.6-fold molar amount and most preferably with a 0.34 to 0.5-fold molar amount, of at least one compound selected from the group consisting of alcohols of the general formula 5 and amines of the general formula 6.

In a further refinement, the method according to the invention additionally provides that astaxanthin of the formula 2 in the organic solvent is reacted with the acid chloride of the general formula 3 in the presence of at least one nitrogen-containing base of the general formula 4; and that the resulting reaction mixture is treated with at least one alcohol of the general formula 5 selected from the group consisting of methanol, ethanol and n-propanol. These primary alcohols are inexpensive to obtain and have the effect that the diester 1 is obtained as a solid due to the removal of by-products described.

A further development of the method according to the invention specifies that astaxanthin of the formula 2 in the organic solvent is reacted with the acid chloride of the general formula 3 in the presence of at least one nitrogen-containing base of the general formula 4; and that the resulting reaction mixture is treated with at least one amine selected from the group consisting of methylamine, ethylamine, n-propylamine, isopropylamine, n-butylamine, sec-butylamine, tert-butylamine, isobutylamine, n-pentylamine, aniline and benzylamine. These amines are also inexpensive to acquire and have the effect that the diester 1 is obtained as a solid due to the removal of by-products described.

The experiments for the conversion and removal of by-products with the aid of the compounds of the general formula 5 and/or 6 showed that it also depends on the duration for which the reaction mixture after the esterification, that is to say, particularly the by-products present therein, are brought into contact with the compounds of the general formulae 5 and/or 6. Nevertheless, the anhydrides present in the reaction mixture and residual acid chlorides of the general formula 3 must react in sufficient amount, if possible completely, with at least one of the compounds of the general formula 5 and/or 6. In order to accommodate this fact, a further elaborated variant of the method according to the invention provides that astaxanthin of the formula 2 in the organic solvent is reacted with the acid chloride of the general formula 3 in the presence of at least one nitrogen-containing base of the general formula 4; and that the resulting reaction mixture is treated with at least one compound selected from the group consisting of alcohols of the general formula 5 and amines of the general formula 6 over a period of 10 min to 3 h, preferably over a period of 20 min to 2 h and most preferably of 30 min to 1 h.

If at least one of the compounds of the general formula 5 or 6 was not added to the reaction mixture after completion of the esterification reaction between astaxanthin of the formula 2 and the acid chloride of the general formula 3, it is, according to the observation of the applicant, scarcely possible to obtain a diester 1 which is sufficiently pure to be crystallized.

Part of the process according to the invention, therefore, is also that the astaxanthin diester of the general formula 1 is generally obtained as a solid, in the course of a crystallization from another organic solvent or a mixture of two or more organic solvents, according to the work-up described.

Therefore, a further aspect of the method according to the invention specifies that astaxanthin of the formula 2 in the organic solvent is reacted with the acid chloride of the general formula 3 in the presence of at least one nitrogen-containing base of the general formula 4; that the resulting reaction mixture is treated with at least one compound selected from the group consisting of alcohols of the general formula 5 and amines of the general formula 6; and that the reaction product of the general formula 1 is crystallized from another solvent or a mixture of two or more solvents.

The further solvent is considered to be any solvent from which the diester 1 can be crystallized. The further solvent is generally alcohols with short alkyl chains, for example methanol, ethanol, n-propanol, isopropanol, n-butanol, sec-butanol, isobutanol, tert-butanol and also the various pentanols, and also cyclopentanol and cyclohexanol. A mixture of two or more solvents is generally understood to mean a mixture of one of the organic solvents with a further solvent. More precisely, as much further solvent is added to the organic solvent with heating such that the diester of the formula 1 is just dissolved.

A further optimized embodiment of the method according to the invention affording good yields specifies that astaxanthin of the formula 2 in dichloromethane is reacted with the acid chloride of the general formula 3 in the presence of at least one nitrogen-containing base selected from the group consisting of N-methylimidazole, pyridine, 3-methylpyridine, 4-dimethylaminopyridine and 5-ethyl-2-methylpyridine; that the resulting reaction mixture is treated with at least one compound selected from the group consisting of methanol, ethanol and n-propanol; and that the reaction product of the general formula 1 is crystallized from an alcohol/ether mixture or from an alcohol/ester mixture.

An alcohol/ether mixture consists of at least one alcohol selected from the group consisting of methanol, ethanol, n-propanol, isopropanol, n-butanol, sec-butanol, isobutanol, tert-butanol and also the various pentanols, and also cyclopentanol and cyclohexanol; and of at least one ether selected from the group consisting of diethyl ether, dipropyl ether, diisopropyl ether, methyl isopropyl ether, t-butyl methyl ether, dibutyl ether, dicyclopentyl ether and cyclopentyl methyl ether.

An alcohol/ester mixture consists of at least one alcohol selected from the group consisting of methanol, ethanol, n-propanol, isopropanol, n-butanol, sec-butanol, isobutanol, tert-butanol and also the various pentanols, and also cyclopentanol and cyclohexanol; and of at least one ester selected from the group consisting of methyl formate, ethyl formate, n-propyl formate, isopropyl formate, n-butyl formate, methyl acetate, ethyl acetate, n-propyl acetate, isopropyl acetate, n-butyl acetate, methyl propionate, ethyl propionate, n-propyl propionate, isopropyl propionate and n-butyl propionate.

If larger amounts of astaxanthin of the formula 2 are reacted, for example on a semi-industrial or industrial scale, larger amounts of hydrochlorides also inevitably result, which are partially soluble, partially insoluble in non-aqueous media. In order nevertheless to be able to remove them completely from the diester of the formula 1, a further variant of the method according to the invention provides that astaxanthin of the formula 2 in the organic solvent is reacted with the acid chloride of the general formula 3 in the presence of at least one nitrogen-containing base of the general formula 4; that the resulting reaction mixture is treated with at least one compound selected from the group consisting of alcohols of the general formula 5 and amines of the general formula 6; and that water is subsequently added to the reaction mixture. The hydrochlorides accumulate completely or virtually completely in the water added and are thus easy to remove from the reaction mixture.

Depending on the method procedure, the reaction mixture is more or less strongly alkaline due to the different bases added. Under basic conditions, esters, such as also the diester of the formula 1, are only moderately stable over an extended period. This is remedied here by a further configuration of the method according to the invention in which the astaxanthin of the formula 2 in the organic solvent is reacted with the acid chloride of the general formula 3 in the presence of at least one nitrogen-containing base of the general formula 4; the resulting reaction mixture is treated with at least one compound selected from the group consisting of alcohols of the general formula 5 and amines of the general formula 6; it is subjected to an acidic work-up; and the reaction product of the general formula 1 is crystallized from another solvent or a mixture of two or more solvents.

The terms “another solvent” and “mixture of two or more solvents” are as have been already defined above.

“Acidic work-up” is understood to mean any type of effect on the reaction mixture which brings said mixture to a neutral or slightly acidic pH. This effect generally means the addition of a Brønsted acid, for example sulfuric acid, hydrochloric acid, phosphoric acid, citric acid, formic acid or acetic acid.

If it is desired to counter the basic character of the reaction mixture and also relatively large batches are employed, the following embodiment of the invention is advantageous. Said embodiment describes a method in which the astaxanthin of the formula 2 in the organic solvent is reacted with the acid chloride of the general formula 3 In the presence of at least one nitrogen-containing base of the general formula 4; the resulting reaction mixture is treated with at least one compound selected from the group consisting of alcohols of the general formula 5 and amines of the general formula 6; water is then added thereto and the mixture is subjected to an acidic work-up; and that the reaction product of the general formula 1 is crystallized from another solvent or a mixture of two or more solvents.

A further aspect of the invention relates to the non-therapeutic use of the diester 1, in which R is a residue selected from the group consisting of C13-C19-alkyl, C13-C19-alkenyl, C13-C19-alkdienyl and C13-C19-alktrienyl, prepared by the method according to the invention, in human or animal nutrition and also in a preparation for human or animal nutrition; preferably diester in which R is a residue selected from the group consisting of C15-C19-alkyl, C15-C19-alkenyl, C15-C19-alkdienyl and C15-C19-alktrienyl; more preferably from the group consisting of C16-C19-alkyl, C16-C19-alkenyl, C16-C19-alkdienyl and C16-C19-alktrienyl; and most preferably diester 1 in which R is a residue selected from the group consisting of C16-C18-alkyl, C16-C18-alkenyl, C16-C18-alkdienyl and C16-C18-alktrienyl.

Furthermore, the invention comprises the diester 1 prepared by the method according to the invention for therapeutic use as a medicament and also as an ingredient for a medicinal preparation; preferably diester 1 prepared by the method according to the invention, in which R is a residue selected from the group consisting of C13-C19-alkyl, C13-C19-alkenyl, C13-C19-alkdienyl and C13-C19-alktrienyl; more preferably from the group consisting of C15-C19-alkyl, C15-C19-alkenyl, C15-C19-alkdienyl and C15-C19-alktrienyl; even more preferably diester 1 prepared by the method according to the invention, in which R is a residue selected from the group consisting of C16-C19-alkyl, C16-C19-alkenyl, C16-C19-alkdienyl and C16-C19-alktrienyl; and most preferably diester 1 prepared by the method according to the invention, in which R is a residue selected from the group consisting of C16-C18-alkyl, C16-C18-alkenyl, C16-C18-alkdienyl and C16-C18-alktrienyl.

Further characteristics, details and advantages of the invention are apparent from the wording of the claims and also from the working examples described below and also comparative examples by reference to the tables and figures. The figures show:

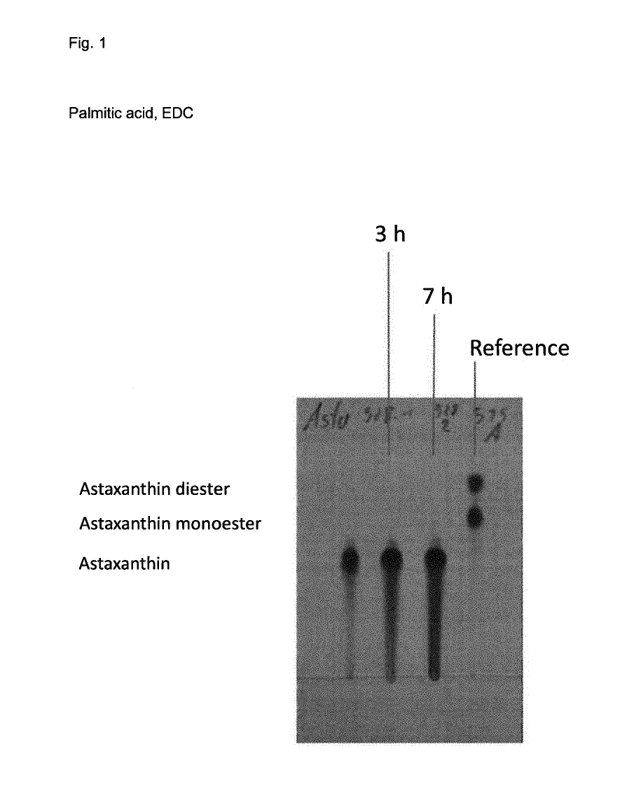

FIG. 1: Thin-layer chromatogram (TLC) of the reaction of astaxanthin 2, palmitic acid, N-(3-dimethylaminopropyl)-N-ethylcarbodiimide hydrochloride (EDC) and N,N-dimethylaminopyridine (DMAP).

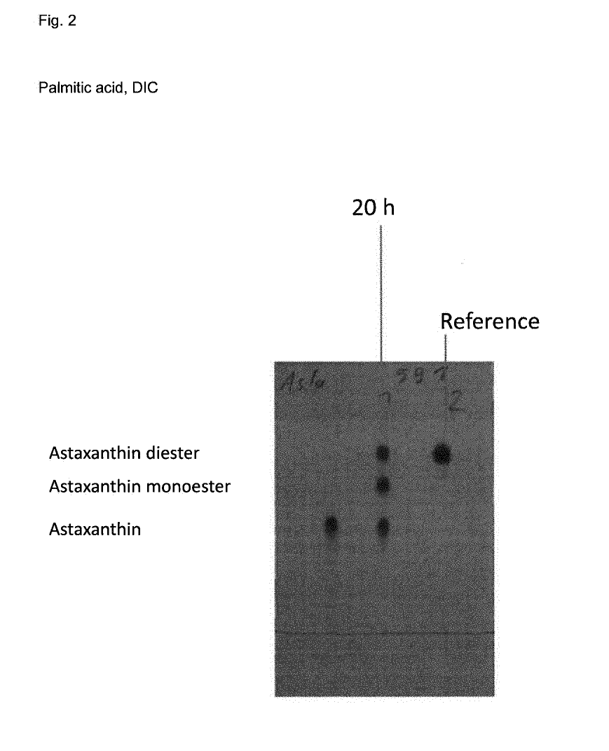

FIG. 2: Thin-layer chromatogram (TLC) of the reaction of astaxanthin 2, palmitic acid, N,N-diisopropylcarbodiimide (DIC) and N,N-dimethylaminopyridine (DMAP).

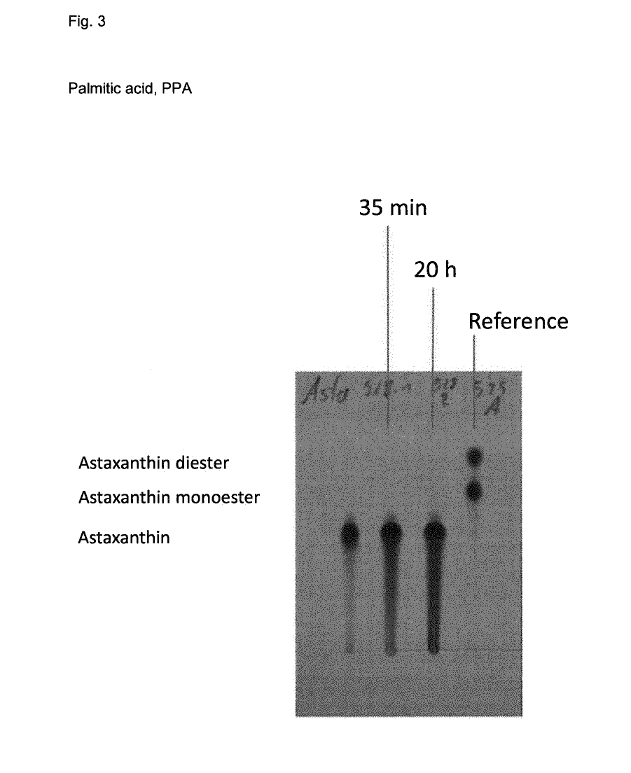

FIG. 3: Thin-layer chromatogram (TLC) of the reaction of astaxanthin 2, palmitic acid, propylphosphonic anhydride and N,N-diisopropylethylamine (DIPEA).

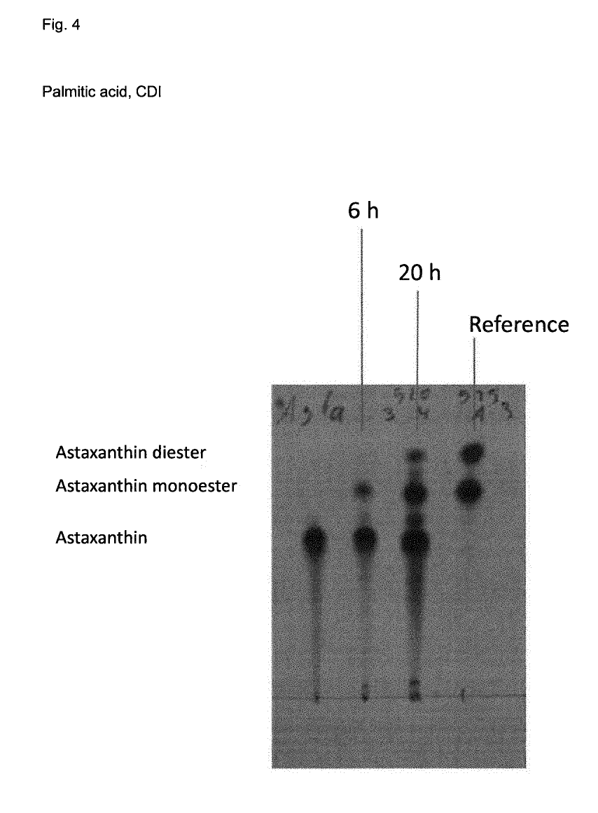

FIG. 4: Thin-layer chromatogram (TLC) of the reaction of astaxanthin 2, palmitic acid, 1,1-carbonyldiimidazole (CDI) and acetic acid.

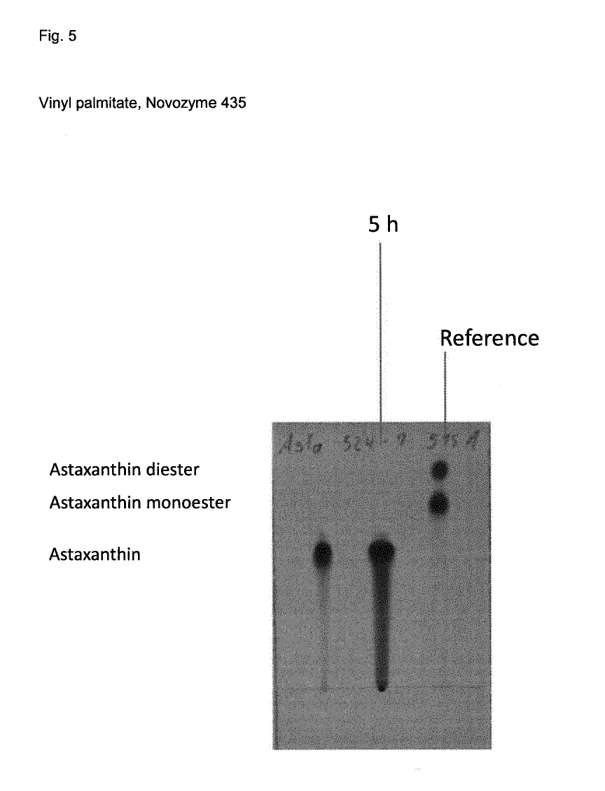

FIG. 5: Thin-layer chromatogram (TLC) of the reaction of astaxanthin 2, vinyl palmitate, Novozyme 435 and acetonitrile.

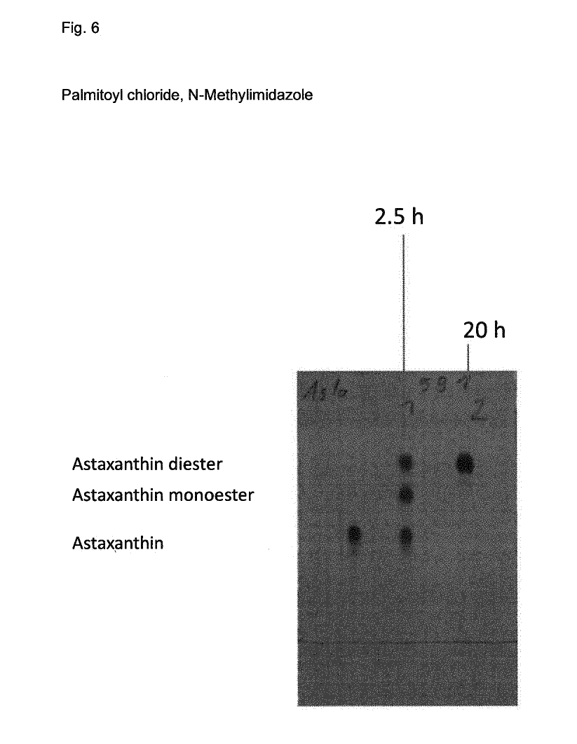

FIG. 6: Thin-layer chromatogram (TLC) of the reaction of astaxanthin 2, palmitoyl chloride and N-methylimidazole.

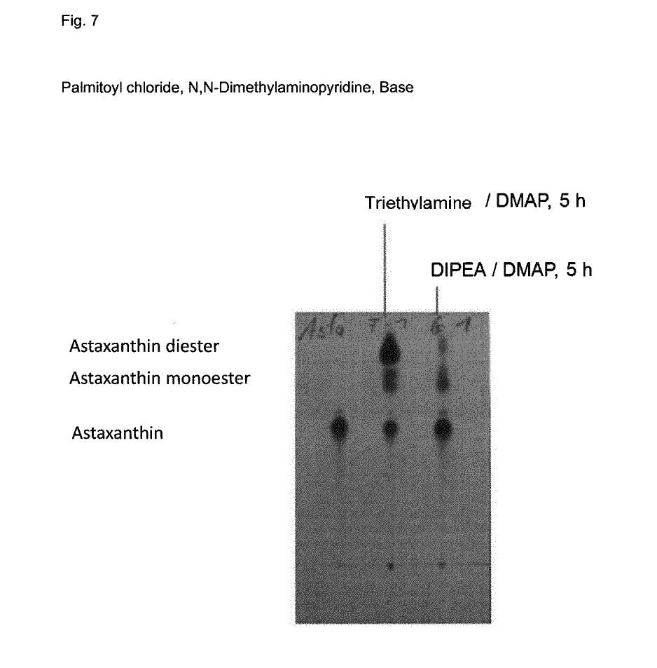

FIG. 7: Thin-layer chromatogram (TLC) of the reaction of astaxanthin 2, palmitoyl chloride, N,N-dimethylaminopyridine (DMAP) and alkylamine base.

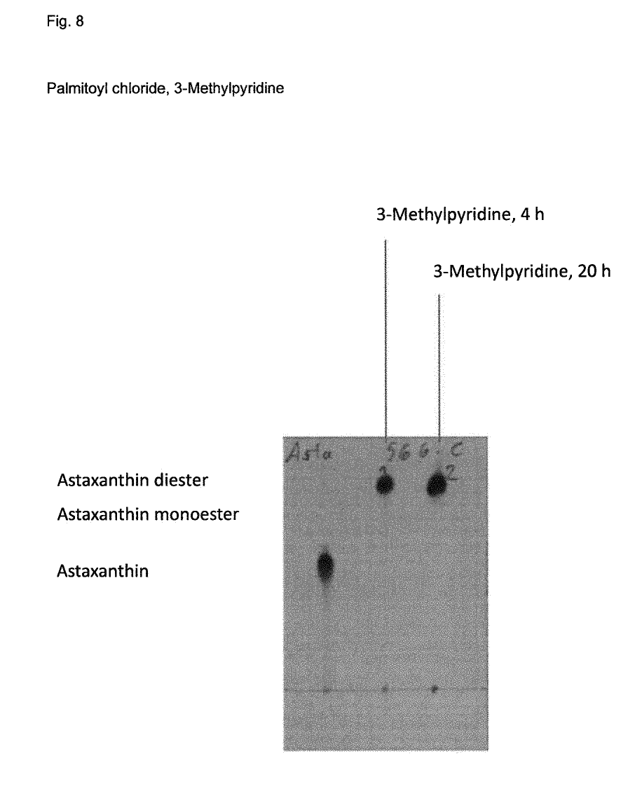

FIG. 8: Thin-layer chromatogram (TLC) of the reaction of astaxanthin 2, palmitoyl chloride and 3-methylpyridine (3-picoline).

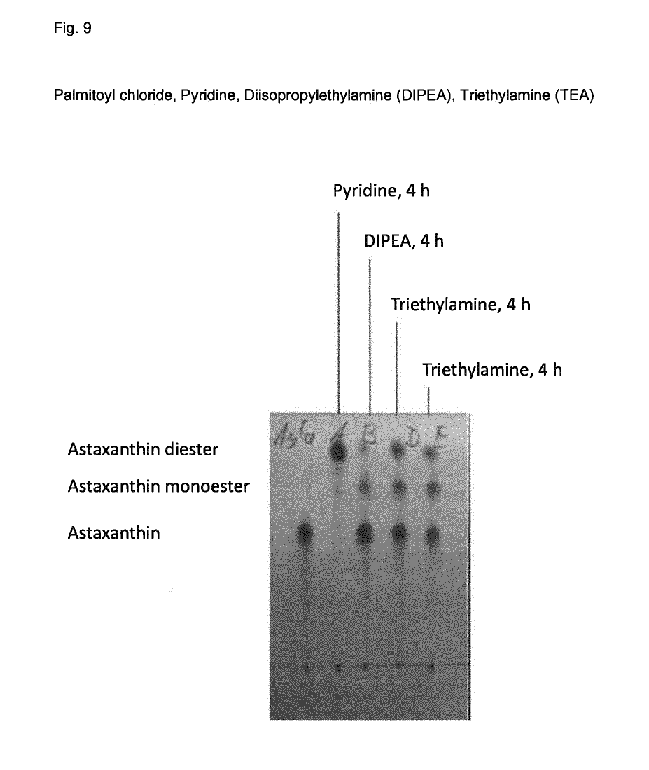

FIG. 9: Thin-layer chromatogram (TLC) of the reaction of astaxanthin 2, palmitoyl chloride, pyridine or diisopropylethylamine (DIPEA) or triethylamine (TEA).

Comparative examples relating to the reaction of astaxanthin 2 with a free carboxylic acid

A free carboxylic acid is understood to mean a carboxylic acid of the general formula 7

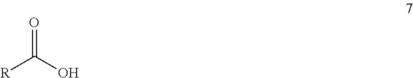

in which R is a residue selected from the group consisting of C9-C19-alkyl, C9-C19-alkenyl, C9-C19-alkdienyl, C9-C19-alktrienyl, where these terms are as already defined in the text above.

### COMPARATIVE EXAMPLE 1: REACTION OF ASTAXANTHIN 2 WITH PALMITIC ACID IN THE PRESENCE OF EDC

3 g (11.7 mmol) of palmitic acid were charged in 47.37 ml (53 g, 740 mmol) of dichloromethane and 3.36 g (17.55 mmol) of N-(3-dimethylaminopropyl)-N-ethylcarbodiimide hydrochloride (EDC) was added at room temperature over 5 minutes. After 2 hours, 3.49 g (5.85 mmol) of astaxanthin 2 was added at room temperature and the mixture stirred at room temperature overnight. The mixture was heated to reflux for 3 hours, then 142.93 mg (1.17 mmol) of 4-dimethylaminopyridine DMAP was added, the mixture boiled under reflux a further 4 hours and then stirred overnight. The conversion to astaxanthin dipalmitate was evaluated by thin-layer chromatography (cyclohexane/ethyl acetate=1:2) and by HPLC.

FIG. 1 shows that no reaction of any sort can be detected after 3 hours and even after 7 hours. Even the formation of astaxanthin monopalmitate, i.e. the corresponding monoester of astaxanthin 2, does not occur.

### COMPARATIVE EXAMPLE 2: REACTION OF ASTAXANTHIN 2 WITH PALMITIC ACID IN THE PRESENCE OF DIC

3 g (11.7 mmol) of palmitic acid were charged in 47.37 ml (53 g, 740 mmol) of dichloromethane and 2.21 g (17.55 mmol) of N,N-diisopropylcarbodiimide (DIC) was added at room temperature over 5 minutes. After 2 hours, 142.93 mg (1.17 mmol) of 4-dimethylaminopyridine (DMAP) and 2.3 g (3.86 mmol) of astaxanthin 2 were added and the mixture heated to reflux for 20 hours. After cooling, the conversion to astaxanthin dipalmitate was evaluated by thin-layer chromatography (cyclohexane/ethyl acetate=1:2).

As can be seen in FIG. 2, even after 20 h large proportions of astaxanthin 2 are unreacted, a further large proportion reacted to give astaxanthin monopalmitate and only a fraction of astaxanthin 2 used formed astaxanthin dipalmitate.

Similar results were obtained when retinoic acid or dihomo-gamma-linolenic acid (DGLA) or gamma-linolenic acid (GLA) were used instead of palmitic acid under otherwise identical conditions.

### COMPARATIVE EXAMPLE 3: REACTION OF ASTAXANTHIN 2 WITH PALMITIC ACID IN THE PRESENCE OF PPA

1.08 g (4.2 mmol) of palmitic acid and 2.39 g (4.0 mmol) of astaxanthin 2 were charged in 25.56 ml (34 g, 400.32 mmol) of dichloromethane. At 0 to 5° C., 3.18 g (5 mmol) of a 50 percent by weight solution of propylphosphonic anhydride solution (PPA) In DMF and then over 3 minutes 1.81 g (14 mmol) of diisopropylethylamine (DIPEA) were added dropwise. The mixture was then stirred for 35 minutes at 0 to 5° C., brought to room temperature and stirred overnight. After said 35 minutes and after 20 hours, the conversion to astaxanthin dipalmitate was evaluated by thin-layer chromatography (cyclohexane/ethyl acetate=1:2).

It can be seen from FIG. 3 that no reaction took place either after 35 minutes or after 20 hours. Not even traces of astaxanthin monopalmitate can be detected after 20 hours.

### COMPARATIVE EXAMPLE 4: REACTION OF ASTAXANTHIN 2 WITH PALMITIC ACID IN THE PRESENCE OF CDI

3 g (11.7 mmol) of palmitic acid were charged in 47.37 ml (53 g, 740 mmol) of dichloromethane. 2.85 g (17.55 mmol) of 1,1′-carbonyldiimidazole (CDI) were added at room temperature in three portions at intervals of 5 minutes each. The mixture was stirred overnight and 3.49 g (5.85 mmol) of astaxanthin 2 were added on the following day. A sample was analyzed by thin-layer chromatography after 6 hours, then 133.8 μl of acetic acid were added and the mixture stirred overnight at room temperature. After 20 hours, a further sample was analyzed by thin-layer chromatography. (Eluent for both chromatograms was cyclohexane/ethyl acetate=1:2.)

FIG. 4 shows that no astaxanthin dipalmitate forms after 6 hours. At best, traces of astaxanthin monopalmitate are detectable. Even after 20 hours, large amounts of unreacted astaxanthin 2 still remain and a certain fraction of astaxanthin monopalmitate is present. The desired astaxanthin dipalmitate can only be detected in very low amounts.

Comparative examples relating to the reaction of astaxanthin 2 with a carboxylic ester

### COMPARATIVE EXAMPLE 5: REACTION OF ASTAXANTHIN 2 WITH VINYL PALMITATE IN THE PRESENCE OF NOVOZYME 435

1.04 g (3.69 mmol) of vinyl palmitate and 1 g (1.68 mmol) of enantiomerically pure 3S,3'S-astaxanthin 2 were charged in 25.45 ml (20 g, 0.49 mmol) of acetonitrile and treated with 1 g of Novozyme 435 (lipase from Candida antarctica Immobilized on acrylic acid resin, CAS Number 9001-62-1, EC Number 232-619-9). This mixture was heated in a water bath to 55° C. (bath temperature 60° C.). A sample was analyzed by thin-layer chromatography after 5 hours at this temperature (eluent: cyclohexane/ethyl acetate=1:2).

It can be seen from FIG. 5 that no conversion of any sort of the enantiomerically pure astaxanthin 2 to astaxanthin monopalmitate or astaxanthin dipalmitate takes place after 5 hours.

A similarly poor result was obtained using vinyl acetate instead of vinyl palmitate under otherwise identical conditions.

Examples relating to the reaction of astaxanthin 2 with an acid chloride

### EXAMPLE 1: REACTION OF ASTAXANTHIN 2 WITH PALMITOYL CHLORIDE IN THE PRESENCE OF METHYL IMIDAZOLE

2.98 g (5 mmol) of astaxanthin 2 were charged in 25 ml (33.25 g, 391.5 mmol) of dichloromethane and 1.32 ml (1.35 g, 16.5 mmol) of N-methylimidazole was added in one portion at room temperature. 4.12 g (15 mmol) of palmitoyl chloride was added dropwise over 2 minutes at 20-28° C. and the heat liberated by the exothermic reaction was removed via an ice bath. A further 25 ml (33.25 g, 391.5 mmol) of dichloromethane were added to the mixture which was stirred at room temperature for 2.5 hours and then stirred overnight. Samples taken after 2.5 hours and after 20 hours were analyzed by thin-layer chromatography (eluent: cyclohexane/ethyl acetate=1:2).

It can be seen in FIG. 6 that even after 2.5 hours a large proportion of astaxanthin 2 has been converted to the corresponding astaxanthin dipalmitate and a further proportion to astaxanthin monopalmitate. After 20 hours, only astaxanthin dipalmitate is found.

### EXAMPLE 2: REACTION OF ASTAXANTHIN 2 WITH PALMITOYL CHLORIDE IN THE PRESENCE OF N,N-DIMETHYLAMINOPYRIDINE (DMAP) AND AN ALKYLAMINE BASE

0.25 g (0.42 mmol) of astaxanthin 2 were charged in 2.09 ml (2.79 g, 30 mmol) of dichloromethane in example 2a and example 2b respectively. In example 2a, 140 mg (192.66 μl, 1.38 mmol) of triethylamine (TEA) and 5.12 mg (0.04 mmol) of N,N-dimethylaminopyridine (DMAP) were added in one portion and likewise in example 2b, 180 mg (240.77 μl, 1.38 mmol) of N,N-diisopropylethylamine (DIPEA) and 5.12 mg (0.04 mmol) of N,N-dimethylaminopyridine (DMAP) were added in one portion. Then, 380 μl (350 mg, 1.26 mmol) of palmitoyl chloride was added in each case in example 2a and example 2b and the mixture was left to stir overnight. The formation of astaxanthin dlpalmitate was investigated by thin-layer chromatography after 5 hours (eluent: cyclohexane/ethyl acetate=1:2).

It can be seen from FIG. 7 that a large proportion of astaxanthin dipalmitate has already been formed after 5 hours using triethylamine (TEA) with catalytic amounts of N,N-dimethylaminopyridine (DMAP) (example 2a), whereas no notable amounts of astaxanthin dipalmitate can be detected after 5 hours using N,N-diisopropylethylamine (DIPEA) and N,N-dimethylaminopyridine (DMAP).

### EXAMPLE 3: REACTION OF ASTAXANTHIN 2 WITH PALMITOYL CHLORIDE IN THE PRESENCE OF 3-METHYLPYRIDINE (3-PICOLINE)

0.25 g (0.42 mmol) of astaxanthin 2 were charged in 2.09 ml (2.79 g, 30 mmol) of dichloromethane. 130 mg (134.51 μl, 1.38 mmol) of 3-methylpyridine were added in one portion. Then, 380 μl (350 mg, 1.26 mmol) of palmitoyl chloride was added and the mixture was left to stir overnight. The formation of astaxanthin dipalmitate was investigated by thin-layer chromatography after 4 hours and 20 hours (eluent: cyclohexane/ethyl acetate=1:2).

FIG. 8 distinctly shows that astaxanthin 2 is already completely converted to astaxanthin dipalmitate after 4 hours and that nothing changes also after 20 hours.

### EXAMPLE 4: REACTION OF ASTAXANTHIN 2 WITH PALMITOYL CHLORIDE IN THE PRESENCE OF PYRIDINE OR DIISOPROPYLETHYLAMINE (DIPEA) OR TRIETHYLAMINE (TEA)

0.25 g (0.42 mmol) of astaxanthin 2 was charged in 2.09 ml (2.79 g, 30 mmol) of dichloromethane for examples 4A, 4B and 4D in each case and in 4.19 ml (5.57 g, 70 mmol) of dichloromethane for example 4E. In example 4A 110 mg (111.34 μl, 1.38 mmol) of pyridine, in example 4B 180 mg (240.77 μl, 1.38 mmol) of N,N-diisopropylamine (DIPEA) and in examples 4D and 4E respectively 140 mg (192.66 μl, 1.38 mmol) of triethylamine (TEA) were added in one portion in each case. Then, 380 μl (350 mg, 1.26 mmol) of palmitoyl chloride was added in each case in all examples and the mixture was left to stir at room temperature. The formation of astaxanthin dipalmitate was investigated by thin-layer chromatography after 4 hours (eluent: cyclohexane/ethyl acetate=1:2).

The second application in FIG. 9 shows a sample from example 4A taken after 4 hours where it can be seen that, after this time, astaxanthin 2 has already completely converted to the corresponding astaxanthin dipalmitate. In example 4B, using diisopropylethylamine (DIPEA) as base, only a low conversion has taken place at this time point. Examples 4D and 4E, using triethylamine (TEA) as base, which differ only in the amount of dichloromethane used as organic solvent, show that astaxanthin dipalmitate has already formed after 4 hours but that the reaction has not yet gone to completion.

### EXAMPLE 5: DETERMINATION OF THE OPTIMAL MOLAR RATIO OF ASTAXANTHIN 2 TO ACID CHLORIDE 3

In examples 5a, 5b, 5c and 5d, 0.4 g (0.67 mmol) of astaxanthin 2 was in each case charged in 3.35 ml (4.46 g, 52.48 mmol) of dichloromethane and 0.17 g (178.51 μl, 2.21 mmol) of pyridine was added in each case. Then, 550 mg (609.99 μl, 2.01 mmol) of palmitoyl chloride was added in example 5a, 520 mg (569.32 μl, 1.89 mmol) of palmitoyl chloride in example 5b, 480 mg (528.66 μl, 1.75 mmol) of palmitoyl chloride in example 5c and 440 mg (487.99 μl, 1.60 mmol) of palmitoyl chloride in example 5d. The mixtures were allowed to react for 5 hours and a sample from each example was analyzed by HPLC under the following conditions

Column: Zorbax Eclipse XDB-C18 1.8 μm 50*4.6 mm from Agilent®

Eluent: -A: 0.05% by volume triethylamine in water
B: tetrahydrofuran

| Time | | Flow rate |
|---|---|---|
| [min] | % B | [ml/min] |
| 0.0 | 40 | 1.2 |
| 8.0 | 100 | 1.2 |
| 10.0 | 100 | 1.2 |
| 10.1 | 40 | 1.2 |

Detector: UV detector λ=470 nm, BW=50 nm
Flow rate: 1.2 ml/min

### Injection: 5 μl

### Temperature: 50° C.

Run time: 12 min

### Pressure: ca. 260 bar

The results are presented in Table 1 below.

TABLE 1

| | | | Astaxanthin |
|---|---|---|---|
| | Astaxanthin | Astaxanthin monopalmitate | dipalmitate |
| | RT 3.2 | RT 5.3 | RT 6.5 |
| Example | [area %] | [area %] | [area %] |
| 5A | 0 | 0.63 | 92.48 |
| 5B | 0.09 | 2.50 | 90.54 |
| 5C | 0.12 | 2.82 | 89.22 |
| 5D | 1.51 | 9.12 | 81.79 |

It can be seen that astaxanthin 2 elutes at a retention time of 3.2 minutes, astaxanthin monopalmitate at a retention time of 5.3 minutes and astaxanthin dipalmitate at a retention time of 6.5 minutes. Example 5a affords the best result. According to the integrated peaks, 92.48% of astaxanthin dipalmitate and 0.63% of astaxanthin monopalmitate are obtained. The astaxanthin 2 starting material is no longer present. Therefore, a particularly good yield of astaxanthin dipalmitate is obtained when the molar ratio of palmitoyl chloride to astaxanthin 2 is 3.

### EXAMPLE 6: SYNTHESIS OF ASTAXANTHIN DIDECANOATE

10 g (16.75 mmol) of astaxanthin 2 and 4.37 g (55.29 mmol) of pyridine are charged in 111.4 g of dichloromethane and 10.65 g (50.26 mmol) of decanoyl chloride are added dropwise at 20° C. over 5 minutes. The reaction mixture is allowed to react overnight, the mixture diluted with 111.4 g of dichloromethane, 0.54 g of methanol and, 30 min later, 16.8 g of water are added and the phases separated. The lower phase is washed with 17.59 g of 10% hydrochloric acid and then twice with 16.75 g of water. The organic phase is rotary evaporated at 50° C., the residue is taken up in ca. 250 ml of t-butyl methyl ether and again fully concentrated. The residue is dissolved in 67 ml of t-butyl methyl ether and 201 ml of ethanol is added dropwise. The mixture is heated to 45° C. and then cooled to 0° C. over 17 h. The precipitated crystalline solid is filtered off under suction, washed twice with 150 ml of ethanol each time and dried at 40° C. in a vacuum drying cabinet. 10.4 g (69% yield) of astaxanthin didecanoate (m.p. 104.8° C.) are obtained.

### EXAMPLE 7: SYNTHESIS OF ASTAXANTHIN DIDODECANOATE

10 g (16.75 mmol) of astaxanthin 2 and 4.37 g (55.29 mmol) of pyridine are charged in 111.4 g of dichloromethane and 12.2 g (50.26 mmol) of dodecanoyl chloride are added dropwise at 20° C. over 5 minutes. The reaction mixture is allowed to react overnight, the mixture diluted with 111.4 g of dichloromethane, 0.54 g of methanol and, 30 min later, 16.8 g of water are added and the phases separated. The lower phase is washed with 17.59 g of 10% hydrochloric acid and then twice with 16.75 g of water. The organic phase is rotary evaporated at 50° C., the residue is taken up in ca. 250 ml of t-butyl methyl ether and again fully concentrated. The residue is virtually dissolved in 117 ml of t-butyl methyl ether at 67° C. and 201 ml of ethanol is added dropwise. The mixture is initially cooled to 45° C. and then to 0° C. over 17 h. The precipitated crystalline solid is filtered off under suction, washed twice with 200 ml of ethanol each time and dried at 40° C. in a vacuum drying cabinet. 11.7 g (73% yield) of astaxanthin didodecanoate (m.p. 130.0° C.) are obtained.

### EXAMPLE 8: SYNTHESIS OF ASTAXANTHIN DIHEXADECANOATE

7.6 g (12.7 mmol) of astaxanthin and 2.98 g (37.7 mmol) of pyridine are charged in 75.9 g of dichloromethane and 9.42 g (34.3 mmol) of hexadecanoyl chloride are added dropwise at 20° C. over 5 minutes. The reaction mixture is allowed to react overnight, the mixture diluted with 75.9 g of dichloromethane, 0.37 g of methanol and, 30 min later, 11.4 g of water are added and the phases separated. The lower phase is washed with 11.4 g of 10% hydrochloric acid and then twice with 11.4 g of water. The organic phase is rotary evaporated at 50° C., the residue is taken up in ca. 217 ml of t-butyl methyl ether and again fully concentrated. The residue is virtually dissolved in 217 ml of ethyl acetate at 50° C. and 108 ml of ethanol is added dropwise. The mixture is initially cooled to 45° C. and then to 0° C. over 17 h. The precipitated crystalline solid is filtered off under suction, washed twice with 72 ml of ethanol each time and dried at 40° C. in a vacuum drying cabinet. 10 g (73% yield) of astaxanthin dihexadecanoate (m.p. 79.7° C.) are obtained.

### EXAMPLE 9: SYNTHESIS OF ASTAXANTHIN DIOCTADECANOATE

10 g (16.75 mmol) of astaxanthin and 4.37 g (55.29 mmol) of pyridine are charged in 111.4 g of dichloromethane and 16.9 g (50.26 mmol) of octadecanoyl chloride are added dropwise at 20° C. over 5 minutes. The reaction mixture is allowed to react overnight, the mixture diluted with 111.4 g of dichloromethane, 0.54 g of methanol and, 30 min later, 16.8 g of water are added and the phases separated. The lower phase is washed with 17.59 g of 10% hydrochloric acid and then twice with 16.75 g of water. The organic phase is rotary evaporated at 50° C., the residue is taken up in ca. 250 ml of t-butyl methyl ether and again fully concentrated. The residue is dissolved in 67 ml of t-butyl methyl ether and 201 ml of ethanol at 53° C. The mixture is cooled to 45° C., seeded and then cooled to 0° C. over 17 h. The precipitated crystalline solid is filtered off under suction, washed twice with 200 ml of ethanol each time and dried at 40° C. in a vacuum drying cabinet. 15.1 g (80% yield) of astaxanthin dioctadecanoate (m.p. 70.5° C.) are obtained.

The method according to the invention is not however, limited to any of the embodiments described above, but is applicable in a variety of ways.

This disclosure presents an environmentally friendly, sustainable and cost-effective method for preparing astaxanthin diesters of the formula 1, in which astaxanthin of the formula 2 is doubly esterified with fatty acid chlorides of the general formula 3. For this purpose, compound 2 and 3 are reacted in an organic solvent in the presence of a nitrogen-containing base of the general formula 4. The invention further relates to the non-therapeutic use of the diester 1, in which R is a residue selected from the group consisting of C13-C19-alkyl, C13-C19-alkenyl, C13-C19-alkdienyl and C13-C19-alktrienyl, in human or animal nutrition and also the therapeutic use of the diester 1 prepared according to the method as a medicament and also as an ingredient in a medicinal preparation.

1. A method for preparing an astaxanthin diester of formula (1) 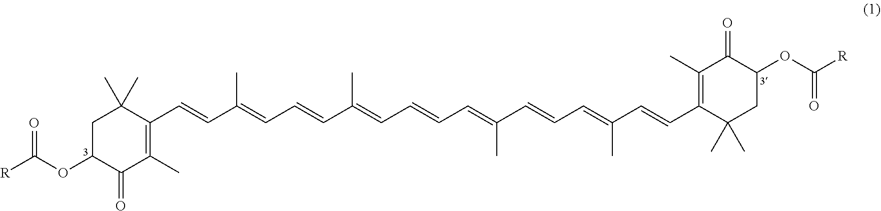
in which the asymmetric center in position 3 and 3′ is racemic, or each has (S) or (R) configuration and R is a residue selected from the group consisting of C9-C19-alkyl, C9-C19-alkenyl, C9-C19-alkdienyl and C9-C19-alktrienyl, wherein
astaxanthin of formula (2) 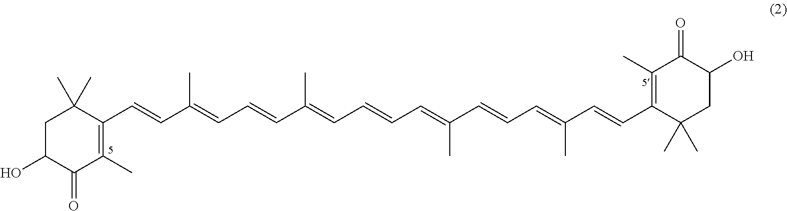 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]
in an organic solvent
is reacted with an acid chloride of formula (3) 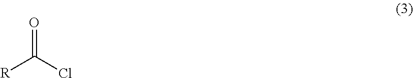
in which R is as defined in formula (1),
in the presence of at least one nitrogen-containing base of formula (4) to provide a reaction product mixture
NR¹R²R³ (4)
in which R¹, R² and R³ are each independently selected from the group consisting of
a saturated C1-C6 chain,
an unsaturated C1-C6 chain,
an aromatic C6 ring,
a C1-C6 chain formed from two of the three residues R¹, R² and R³, wherein said two residues are linked to each other and, together with the nitrogen atom of the base (4), form an alkylated or non-alkylated heterocycle or an alkylated or non-alkylated heteroaromatic cycle, or
a C1-C6 chain formed from two of the three residues R¹, R² and R³, wherein said two residues are linked to each other via a further nitrogen atom and, together with the nitrogen atom of the base (4), form an alkylated or non-alkylated heterocycle or an alkylated or non-alkylated heteroaromatic cycle.

2. The method according to claim 1, wherein the astaxanthin of the formula (2) in the organic solvent is reacted with a greater than two-fold molar excess, based on astaxanthin (2), of the acid chloride of the formula (3) in the presence of at least one nitrogen-containing base of the formula (4).

3. The method according to claim 1, wherein the astaxanthin of the formula (2) in the organic solvent is reacted with a 2.3-fold to 7-fold molar excess, of the acid chloride of the formula (3) in the presence of at least one nitrogen-containing base of the formula (4).

4. The method according to claim 1, wherein the organic solvent is a chlorine-containing organic solvent selected from the group consisting of dichloromethane, trichloromethane, tetrachloromethane, 1,1-dichloroethane, 1,2-dichloroethane, trichloroethylene, tetrachloroethylene, perchloroethylene, chlorobenzene or a mixture of at least two of the chlorine-containing organic solvents.

5. The method according to claim 4, wherein the astaxanthin of the formula (2) in the organic solvent is reacted with the acid chloride of the formula (3) at a temperature range of −20 to +100° C.

6. The method according to claim 1, wherein the at least one nitrogen-containing base of the formula (4) is selected from the group consisting of monocyclic nitrogen-containing bases, and bicyclic nitrogen-containing bases.

7. The method according to claim 1, wherein the present in a 1.1 to 2-fold molar ratio, based on the acid chloride.

8. The method according to claim 1, wherein the reaction product mixture is treated with at least one compound selected from the group consisting of alcohols of the formula (5)
R⁴OH (5)
where R⁴ is equal to C1-C6-alkyl; and amines of the formula (6)
R⁵R⁶NH (6)
where R⁵ and R⁶ are each independently equal to H or C1-C6-alkyl, in which R⁵ and R⁶ either each form an independent group or are linked to each other.

9. The method according to claim 8, wherein the reaction product mixture is treated with a molar deficiency, based on the amount of acid chloride (3), of at least one compound selected from the group consisting of alcohols of the formula (5) and amines of the formula (6).

10. The method according to claim 9, wherein the reaction product mixture is treated with a 0.2 to 0.7-fold molar amount, based on the amount of acid chloride (3), of at least one compound selected from the group consisting of alcohols of the formula (5) and amines of the formula (6).

11. The method according to claim 8, wherein the at least one alcohol of the formula (5) is selected from the group consisting of methanol, ethanol and n-propanol.

12. The method according to claim 8, wherein the reaction mixture treated with at least one compound selected from the group consisting of alcohols of the formula (5) and amines of the formula (6), is treated over a period of 10 min to 3 h.

13. The method according to claim 8, further comprising recrystallizing the astaxanthin diester of formula (1) from another solvent or a mixture of two or more solvents.

14. The method according to claim 8, further comprising adding water to the reaction product mixture following treatment with the at least one compound selected from the group consisting of alcohols of the formula (5) and amines of formula (6).

15. The method according to claim 14, wherein following the addition of the water, the reaction product mixture is subjected to an acidic work-up; and the reaction product of the formula (1) is crystallized from another solvent or a mixture of two or more solvents.

16. (canceled)

17. An astaxanthin diester of formula (1) for therapeutic use as a medicament, or as an ingredient for a medicinal preparation, in which R is a residue selected from the group consisting of C13-C19-alkyl, C13-C19-alkenyl, C13-C19-alkdienyl and C13-C19-alktrienyl.
